(19) United States
(12) Patent Application Publication
(10) Pub. No.: US 2014/0342467 A1
(43) Pub. Date: Nov. 20, 2014

(54) APPARATUS AND METHOD FOR CONTINUOUSLY MONITORING SUBAQUEOUS TARGET HARMFUL SUBSTANCES

(71) Applicant: KOREA INSTITUTE OF SCIENCE AND TECHNOLOGY, Seoul (KR)

(72) Inventors: Yeon Seok Kim, Seoul (KR); Jong Soo Jurng, Seoul (KR); Byoung Chan Kim, Seoul (KR); Hyoun Duk Jung, Seoul (KR); Jin Young Kim, Seoul (KR)

(73) Assignee: KOREA INSTITUTE OF SCIENCE AND TECHNOLOGY, Seoul (KR)

(21) Appl. No.: 13/946,656

(22) Filed: Jul. 19, 2013

(30) Foreign Application Priority Data
May 16, 2013 (KR) 10-2013-0055411

(51) Int. Cl. G01N 33/18

(52) U.S. Cl. CPC G01N 33/1813; G01N 33/1886
USPC 436/501; 422/69; 435/288.1

(57) ABSTRACT

The present invention relates to an apparatus and method for continuously monitoring subaqueous target harmful substances. More particularly, it relates to an apparatus and method for continuously monitoring subaqueous target harmful substances by continuously measuring the concentration of the subaqueous target harmful substances. The present invention provides an apparatus and method for continuously monitoring subaqueous target harmful substances, which can continuously measure the concentration of subaqueous target harmful substances using a receptor that can selectively recognize the target harmful substances, a porous membrane fixed with the receptor, and a sensing unit that continuously measures the intensity of fluorescent signals of the target harmful substance reacting with the receptor, and can be utilized as various apparatuses and methods for continuously sensing various harmful substances necessary to continuously monitor for the management of the water quality.

### CROSS-REFERENCE TO RELATED APPLICATION

This application claims under 35 U.S.C. §119(a) the benefit of Korean Patent Application No. 10-2013-0055411 filed May 16, 2013, the entire contents of which are incorporated herein by reference.

### BACKGROUND

(a) Technical Field

The present invention relates to an apparatus and method for continuously monitoring subaqueous target harmful substances. More particularly, it relates to an apparatus and method for continuously monitoring subaqueous target harmful substances by continuously measuring the concentration of the subaqueous target harmful substances.

(b) Background Art

Generally, it is very important to supply clean water to people by strictly regulating and managing the concentration of harmful substances present in various water systems such as public water supply, waste water, and river. To this end, it is necessary to keep monitoring the presence of harmful substances.

While instrumental analysis methods such as HPLC, CG/MS, and AA used to measure subaqueous harmful substances are highly accurate and sensitive detection, expensive equipments, professionally skilled persons and samples containing harmful substances should be provided in an analysis room, thus requiring much time and cost.

To resolve the limitations, portable and cheap detection units and methods using sensors based on functional organic/inorganic materials or based on bioreceptors such as antibodies, enzymes, and aptamers have been under development, but these sensor-typed detector units are mostly not high in sensitivity despite high convenience and portability.

Particularly, one of most important issues in monitoring subaqueous harmful substances is that it is possible to continuously measure harmful substances in all of instrumental analysis methods and methods using sensor technology.

As examples of a related art, Korean Patent Application Publication No. 10-2010-0088932 (Aug. 11, 2010) discloses a multiple water monitoring sensor, and Korean Patent No. 10-0337943 (May 13, 2002) discloses a multi-channel device for continuously monitoring toxicity in water and method for monitoring toxicity in water using same. Also, Korean Patent Application Publication No. 10-2012-0101927 (Sep. 17, 2012) discloses a heavy metal and harmful substance detecting sensor chip using aptamer modifying light emitting polymer and measuring device using thereof.

However, these related arts have limitations in that continuous measurement of specific harmful chemical substances and measurement of harmful substance concentration are impossible.

Also, the reason why A Tele-Monitoring System (TMS) used in inspecting the water quality in real time measures only items such as pH, turbidity, temperature and conductivity except the concentration of harmful substances that are important factor of water pollution is that it is very difficult to continuously measure the concentration of harmful substances in terms of technology.

At present, samples intermittently taken at a certain time interval are transferred to a laboratory, where the samples are preprocessed and then analyzed to accumulate data. Similarly, microorganisms in a water system are also regularly sampled and cultured in a laboratory, but the analysis period takes one to three days, somewhat long.

Accordingly, it is difficult to quickly deal with water pollution, and thus the analysis results about harmful substances can be handled in terms of post-management.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention provides an apparatus and method for continuously monitoring subaqueous target harmful substances, which can continuously measure the concentration of subaqueous target harmful substances using a receptor that can selectively recognize the target harmful substances, a porous membrane fixed with the receptor, and a sensing unit that continuously measures the intensity of fluorescent signals of the target harmful substance reacting with the receptor.

In one aspect, the present invention provides an apparatus for continuously monitoring subaqueous target harmful substances, including: a porous membrane mounted in a hollow column tube; a receptor fixed on the porous membrane and reacting with the subaqueous target harmful substances; and a sensing unit continuously measuring a signal generated and accumulated by the reaction between the receptor and the target harmful substances.

In another aspect, the present invention provides a method for continuously monitoring subaqueous target harmful substances, including: manufacturing a receptor complex in which a target substance selective DNA receptor and a complementary DNA complementarily combining with the target substance selective DNA receptor are combined in a double helix structure; fixing the receptor complex on a porous membrane; continuously passing a solution containing a target substance through the porous membrane on which the receptor complex is fixed and then continuously measuring a fluorescence signal generated by a reaction between the receptor and the target substance on a surface of the membrane; and detecting an instantaneous concentration of the target substance in the sample solution that is introduced, by analyzing a variation of the fluorescence signal that is continuously measured.

Other aspects and exemplary embodiments of the invention are discussed infra.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a view illustrating an apparatus and method for continuously monitoring subaqueous target harmful substances according to an embodiment of the present invention;

FIG. 2 is a graph illustrating fluorescent signals and concentration of target harmful substances measured by an apparatus and method for continuously monitoring subaqueous target harmful substances according to an embodiment of the present invention;

FIG. 3 is a view illustrating fluorescence images obtained by passing a mercury ion solution through a membrane fixed with a receptor at different flow rates using an apparatus and method for continuously monitoring subaqueous target harmful substances according to an embodiment of the present invention;

FIGS. 4A and 4B are views illustrating fluorescence images and fixel density obtained by passing a mercury ion solution through a membrane fixed with a receptor at a constant flow rate using an apparatus and method for continuously monitoring subaqueous target harmful substances according to an embodiment of the present invention;

FIGS. 5A and 5B are views illustrating fluorescence images and fixel density obtained by passing mercury ion solutions of different concentrations and water through a membrane fixed with a receptor at a constant flow rate for a certain time, using an apparatus and method for continuously monitoring subaqueous target harmful substances according to an embodiment of the present invention.

Reference numerals set forth in the Drawings includes reference to the following elements as further discussed below:
10: column tube
12: membrane
14: receptor
16: sensing unit
18: container

It should be understood that the accompanying drawings are not necessarily to scale, presenting a somewhat simplified representation of various exemplary features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The above and other features of the invention are discussed infra.

Hereinafter, exemplary embodiments of the present invention will be described in detail with reference to the accompanying drawings.

The present invention provides a sensing unit including a porous membrane fixed with a receptor selectively combining with subaqueous target harmful substances and a method for continuously monitoring target substances using the sensing unit.

Unlike a sensor type of measurement apparatuses that generally performs single measurement, the apparatus for monitoring subaqueous target substances according to the embodiment continuously measures signals created and accumulated by a reaction between a receptor and target substances by continuously passing a receptor capable of recognizing target substances through a porous membrane, and measure in real-time the concentration of subaqueous target substances passing through the membrane based on the measured results.

In this case, signals generated by the reaction between the receptor and the target substances may steadily increase or decrease according to the accumulated amount of target substances among an aqueous sample, and there is a correlation between the change degree of the signal and the concentration of a target substance existing in the sample at each time.

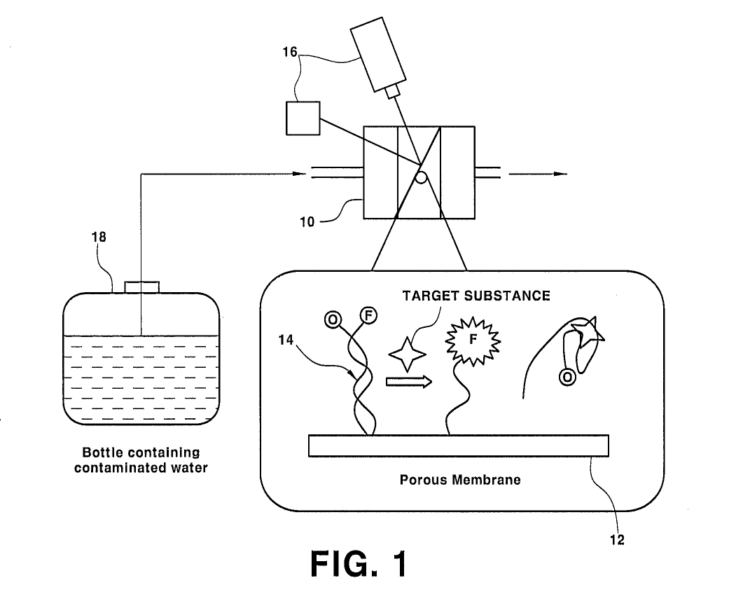

Hereinafter, a configuration of an apparatus for continuously monitoring subaqueous target harmful substances according to an embodiment of the present invention will be described with reference to FIG. 1.

The apparatus for monitoring subaqueous target harmful substances may include a porous membrane 12 mounted in a hollow column tube 10 having a certain diameter, a receptor 14 fixed on the porous membrane 12 to react with subaqueous target harmful substances, and a sensing unit 16 for continuously measuring signals generated and accumulated by a reaction between the receptor 14 and the target harmful substances.

Also, the inlet of the hollow column tube 10 mounted with the membrane 12 on which the receptor 14 is fixed may be connected to a container 18 containing an aqueous sample via a tube.

Although not shown in FIG. 1, the monitoring apparatus may include a flow rate control pump that supplies a sample solution in the container 16 to the membrane 12 inside the hollow column tube 10 at a certain flow rate.

In this case, the sensing unit 16 may include all types of sensors that can sense electrochemical signals, electrical signals, optical signals such as a color change, and fluorescence signals generated and accumulated by the reaction between the receptor 14 and the target harmful substances.

The porous membrane 12 may be formed of silica, cellulose, polymer, and metal foam, but may be formed of all types of materials that can pass the sample solution as well as fix the receptor. In the following embodiments, a porous membrane formed of a silica material will be used as the membrane.

The receptor 14 may include all types of receptors that can selectively recognize biomaterials such as functional DNA, aptamer, antibody, and enzyme, and specific subaqueous target harmful substances such as functional polymer and organic/inorganic materials.

Even though any type of receptor that can selectively recognize target harmful substances can be used, in this embodiment, as the mercury ion that is one of main indicators is selected as a model target harmful substances, a functional single-stranded DNA that can selectively combine with the mercury ion will be selected as a model receptor.

More specifically, the functional single-stranded DNA, i.e., mercury ion selective single-stranded DNA that can selectively combine with the mercury ion used in this embodiment includes T-rich ssDNA in which a lot of thymine of DNA sequence is included. Many previous researches show that the functional single-stranded DNA has a very high selectivity with respect to the mercury ion (Angew. Chem. Int. Ed. 46 (2007) 4093-4096; Chem. Soc. Rev. 40 (2011) 5855-5866).

As described below, the receptor 14 may be used as a receptor complex (double helix DNA shown in FIG. 1) including a double helix DNA in which the target harmful substance selective DNA receptor and its complementary DNA are combined.

Hereinafter, an example of manufacturing an apparatus for continuously monitoring subaqueous target harmful substances and a method of continuously monitoring target harmful substances according to an embodiment of the present invention will be described as follows.

a) First, the target harmful substance selective DNA receptor and its complementary DNA are combined to form a receptor complex including the double helix DNA (a).

b) Next, the surface functionalization of the silica membrane is performed for the fixation of the receptor (b), and then the receptor complex is fixed on the porous membrane (c).

d) Next, a solution containing a target harmful substance is continuously passed through the membrane, and then a fluorescence signal generated by a reaction between the receptor and the target harmful substance are continuously measured (d). The variation of the fluorescence signal that is continuously measured is analyzed to detect the instantaneous concentration of the target harmful substance in the sample solution that is introduced (e).

Particularly, the target harmful substance selective receptor DNA among DNAs used in step (a) is marked with a fluorescence inhibiting substance (acceptor), and the DNA complementary to the receptor DNA is marked with a fluorescence material (donor) and a functional group at both sides thereof.

Hereinafter, the example of manufacturing an apparatus for continuously monitoring subaqueous target harmful substances and the method of continuously monitoring target harmful substances according to the embodiment of the present invention will be described in more detail as follows.

(1) Manufacturing of Target Substance Selective Double Helix DNA Receptor Complex

To manufacture a receptor, the target harmful substance selective DNA receptor and its complementary DNA are combined to form a receptor complex including the double helix DNA.

A single-stranded functional DNA or aptamer is used as the target harmful substance selective DNA receptor that can selectively recognize a target harmful substance, and one end of the single-stranded DNA receptor is marked with an inhibiting substance that can inhibit a fluorescence signal.

Also, a DNA having a complementary nucleotide sequence with respect to the single-stranded DNA receptor is used for the complementary DNA that can be complementarily combined with the target harmful substance selective DNA receptor.

In this case, the DNA (hereinafter, referred to as complementary DNA) having the complementary nucleotide sequence to the single-stranded DNA receptor (hereinafter, referred to as DNA receptor) has both ends functionalized with a fluorescence material that can emit a fluorescence and an amine group for fixation on the membrane.

Next, in order to manufacture the double helix DNA by combining the two complementary single-stranded DNAs, i.e., the single-stranded DNA receptor and the DNA having the complementary nucleotide sequence to the single-stranded DNA receptor, the receptor DNA and the complementary DNA are put in a phosphate buffer including NaCl, and then react at a temperature of about 35° C. to about 42° C. for about 5 hours.

Thus, when the double helix DNA receptor, i.e., the receptor complex is manufactured, the fluorescence emitted from the fluorescence material marked on the end of the complementary DNA is first inhibited from emitting to the outside by an inhibiting substance (quencher) marked on the end of the receptor DNA such that the fluorescence signal can be smoothly measured as described below.

Finally, in order to check whether or not the combination of the receptor DNA and the complementary DNA is successful, the intensities of the fluorescence before and after the combination reaction may be measured and compared to each other.

(2) Functionalization of Membrane Surface and Fixation of Receptor Complex

As a material for the porous membrane that can fix the DNA receptor and easily pass the solution, various materials such as silica, cellulose, polymer, and metal foam can be used, and the method for fixing the receptor can be changed according to the material of the membrane.

Herein, the method of fixing the DNA receptor will be described as using a silica membrane as the membrane.

In order to fix the receptor complex, i.e., double helix DNA receptor on the surface of the silica membrane, the surface of the membrane needs to be first chemically functionalized.

For this, the silica membrane is first functionalized using aminosilane.

In this case, for the fixation of the DNA receptor functionalized with an amine group, the amine-treated surface of the membrane is functionalized with a chemical substance with a carboxyl group at both ends, and after a coupling material is treated, and then the DNA receptor complex with an amine group is treated and fixed.

Also, when the DNA receptor having an end functionalized with carboxylic acid, the aminosilane-treated surface of the membrane is activated with the coupling material, and then the receptor is immediately injected and fixed.

Finally, the surface of the membrane on which the receptor is not fixed may be treated with a blocking solution to minimize an non-specific adsorption.

Thus, after the fixation is completed, chemical substances remaining on the surface of the membrane is completely removed using distilled water. In order to optimize the method of fixing the DNA receptor on the surface of the membrane, only the complementary DNA marked with a fluorescent material is fixed on the surface of the membrane, and then the intensity of a fluorescence signal generated on the surface of the membrane is measured.

(3) Continuous Injection of Aqueous Sample and Signal Measurement

First, as shown in FIG. 1, Also, a tube is connected to the inlet of the hollow column tube 10 mounted with the membrane 12 on which the receptor 14 is fixed, and one end of the tube is dipped in the container 18 containing an aqueous solution including a target substance.

Next, the intensity of the fluorescence generated on the surface of the membrane is measured using the sensing unit 16 while continuously injecting a target substance solution with a certain concentration by a pump, or sequentially injecting a target substance solution with different concentrations or water without a target substance.

In this case, the aqueous solution needs to be maintained at a uniform flow rate. Since the optimal flow rate varies with the state of the membrane, the physical/chemical characteristics of the target substance, and the type of the receptor, the optimal flow rate needs to be changed according to the system.

When the flow rate is too high, a time taken for the target substance to react with the receptor on the surface of the membrane while target substance is passing through the membrane may not be sufficient, and thus an obtained signal value may be lowered. In contrast, when the flow rate is too low, the reaction between the target substance and the receptor may be sufficiently performed, but it is difficult to measure a large amount of sample and the increase of the accumulated signal is slow. Accordingly, the flow rate of the aqueous solution needs to be uniformly controlled according to the state of the membrane, the physical/chemical characteristics of the target substance, and the type of the receptor.

(4) Method of Analyzing in Real-Time Instantaneous Concentration of Target Substance in Sample from Accumulated Signal

As described above, when the aqueous solution including the target substance passes through the membrane, the receptor DNA (Q of FIG. 1) combined with the complementary DNA (F of FIG. 1) fixed on the membrane in a double helix structure combines with the target substance to be separated from the complementary DNA, and then passes through the membrane together with the target substance.

In this case, since the inhibiting substance that inhibits the fluorescence expression of the fluorescent material at the end of the complementary DNA is separated and lost together with the complex in which the receptor DNA and the target substance are combined, the fluorescent material expression at the end of the complementary DNA is recovered, and thus the fluorescence signal increases on the surface of the membrane.

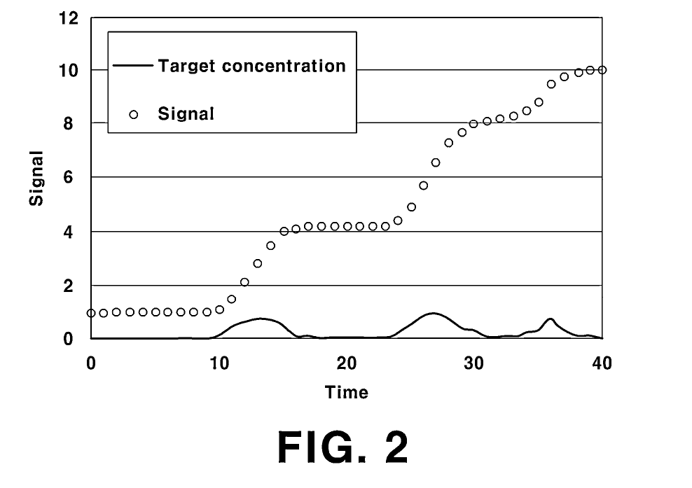

Accordingly, as shown in FIG. 2, the fluorescence signal is accumulated and increased in proportion to the amount of the target substance passing through the membrane. Also, as the concentration of the target substance in the sample solution increases, the increase speed of the fluorescence signal also increases.

However, when there is no target substance in the solution sample, the fluorescence signal does not increase.

The intensity of the fluorescence signal is continuously measured at a certain time interval. When an actual sample with unknown concentration is continuously monitored, the concentration of the target substance in the sample at the moment when the target substance passes through the membrane can be expressed as a function of a change speed of the accumulated fluorescence signal.

In this case, when the intensity of the fluorescence signal is saturated, the membrane and the receptor are replaced.

Hereinafter, the present invention will be described in more detail through examples. However, since the examples below are just for exemplifying the present invention, the scope of the present invention should not be construed as limited to these examples.

### EXAMPLES

The following examples illustrate the invention and are not intended to limit the same

The following method was used to manufacture a porous membrane with a DNA receptor fixed thereon, which can selectively combine with a mercury ion solution.

First, A mercury ion specific DNA receptor (R-DNA) was marked with a fluorescence inhibiting substance at 3′ end, and a DNA (C-DNA) having a complementary nucleotide sequence with respect to the receptor was marked with a fluorescent material and an amine group at the 5′ terminus and 3′ terminus, respectively (GenoTech Inc.).

R-DNA and C-DNA have the following nucleotide sequence.

R-DNA:
5′-TTCTTTCTTCCCTTGTTTGTT-Dabcyl-3′,
C-DNA:
5′-FAM-AAGAAAGAAGGGAACAAACAA-C7-NH2-3′.

For the hybridization of the two DNAs, 50 ul R-DNA and C-DNA of 2 uM, respectively, were put in a 400 ul phosphate buffer solution (PBS, pH 7.4) including 1M NaCl, and then were mixed at a temperature of about 35° C. to about 42° C. for about five hours.

The fluorescence signal value of the solution before and after being mixed were about 842.3 and 4.2, respectively. It was confirmed that most R-DNA and C-DNA were combined. At this point, the intensity of the fluorescence signal was analyzed by a fluorescence spectroscope (LS50B, PerkinElmer).

As a membrane for fixing the R/C-dsDNA receptor complex, a column unit (Quick plasmid mini column) included in a plasmid DNA purification kit (PureLink Quick Plasmid Miniprep kit) from Invitrogen Inc. was used. This unit includes a 750 ul volume of column and a silica membrane.

In order to fix a DNA functionalized with an amine group on the surface, 1 to 5% aminosilane APTES, (3-Aminopropyl)triethoxysilane in anhydrous toluene) was first dropped on the silica membrane and treated for about 24 hours or more.

Also, the membrane were twice washed using a pump in the order of the anhydrous toluene and distilled water and ethanol and distilled water to remove the aminosilane substance adsorbed to the membrane.

Next, a coupling material (Sulfo-NHS and EDC) was put for a reaction for about 1 to 2 hours, and then was removed through centrifugation.

Next, in order to functionalize the surface of the membrane with a carboxylic group, a 1 to 5% succinic acid solution having the carboxylic group at both sides was treated, and a reaction was performed for about 24 hours or more.

Next, the membrane was again washed with distilled water, and then the coupling material (Sulfo-NHS and EDC) was again put for a reaction for about 1 to 2 hours.

Finally, a 1 uM, 100 ul R/C-dsDNA receptor complex marked with an amine group was injected for a reaction for about at least 2 hours, and the activated carboxylic group remaining on the surface of the membrane after the reaction with the DNA was blocking-treated using a 0.1 M ethanolamine solution. The membrane is finally washed with distilled water.

Meanwhile, in order to check the efficiency of the method for fixing the DNA receptor, C-DNA in which the fluorescence is not inhibited was fixed on the membrane in the same manner.

When the fluorescence intensity of the solution passing through the membrane through centrifugation after the fixation treatment of C-DNA was measured by the fluorescence spectroscope, the fluorescence intensity corresponded to about 37.5% of the fluorescence intensity of the original C-DNA solution injected to the membrane. Accordingly, it could be verified indirectly through the mass correction that about 60% of the injected C-DNA was fixed on the surface of the membrane.

In order to more directly verify that the receptor DNA was fixed on the surface of the membrane, the fluorescence intensity of the surface was compared with that of a membrane treated only C distilled water.

First, the membrane was recovered from the column unit, and then put on an ultraviolet irradiator (ECX-F20.M, VILBER LOURMAT) to acquire a fluorescence image of the membrane using a digital camera (Canon G12, ISD sensitivity: 100).

The fluorescence intensity of the acquired fluorescence image was analyzed using an image analysis program (ImageJ). The fluorescence intensity of the membrane treated only with distilled water was about 25.8, which showed a basic value. The fluorescence intensity of the membrane with 1 uM, 100 ul C-DNA fixed thereon showed a relatively high value of about 160.5, showing that the receptor DNA was sufficiently fixed on the surface of the membrane.

### Test Example 1

A test was performed to verify whether or not the continuous passing of mercury ion through the membrane can effectively generate the actual fluorescence signal before a continuous mercury ion monitoring test is performed using a porous membrane with a receptor fixed thereon as described above.

Similarly to the example described above, after the receptor complex was fixed on the silica membrane, the fluorescence intensity on the surface of the membrane was measured and compared between a case where distilled water, 100 ppm 500 ul mercury ion solution, and 1 ppm 500 ul mercury ion solution were put into the column to pass through the membrane and a case where 1 ppm 50 ml mercury ion solution passed through the membrane at different flow rates of about 1, 5, 10, and 50 ml/min, respectively.

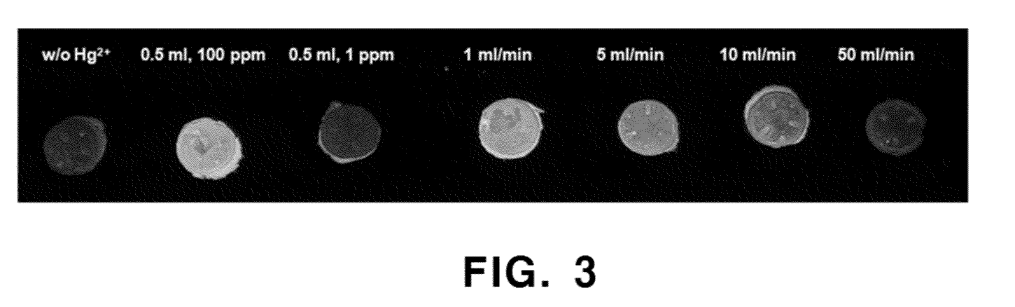

As a result, as shown in the first and third images from the left side of FIG. 3, when distilled water and 1 ppm 500 ul mercury ion solution passed through the membrane, very weak fluorescence images were obtained. As shown in the second image, when 100 ppm 500 ul mercury ion solution passed through the membrane, a relatively strong fluorescence image was obtained.

Also, as shown in the fourth to seventh images from the left side of FIG. 3, when 1 ppm, 50 ml mercury ion solution passed through the membrane at an increasing flow rate, the flow rate increases in the order of 1, 5, 10, and 50 ml/min. Accordingly, it could be seen that the intensity of the fluorescence image was shown as weak. This is probably because the reaction time was not sufficient between the mercury ions included in the solution and the mercury ion receptor on the surface of the membrane due to too fast movement speed of the mercury ions.

Also, when the mercury ion solution flows at a flow rate of about 1 ml/min, the fluorescence intensity was about 95% compared to the fluorescence intensity of the membrane in case where 100 ppm, 0.5 ml mercury ion solution passed through the membrane, acquiring a strong fluorescence image by allowing the same amount of mercury ion (50 ug) to react with the mercury ion receptor on the surface of the membrane. Thus, even when the sample solution passes through the membrane at a slow flow rate of about 1 ml/min, it is estimated that the reaction time between the target substance and the receptor is sufficient.

### Test Example 2

A test was performed to verify whether or not the fluorescence signal linearly increases in proportion to the amount (or time) of mercury ion solution passing through the membrane when the mercury ion solution with a certain concentration passes through the membrane at a certain flow rate.

Similarly to the example described above, the receptor complex was fixed on the silica membrane, and then 1 ppm mercury ion solution passed through the membrane at a flow rate of about 5 ml/min for about 1, 5, 10, and 30 minutes, respectively. Thereafter, the fluorescence intensity of the membrane surface was analyzed.

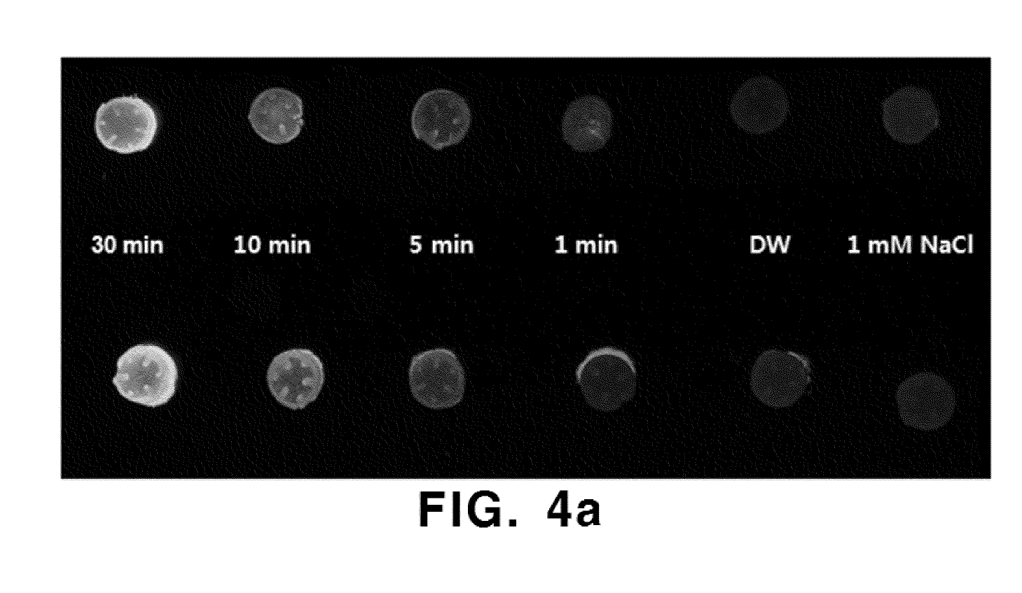

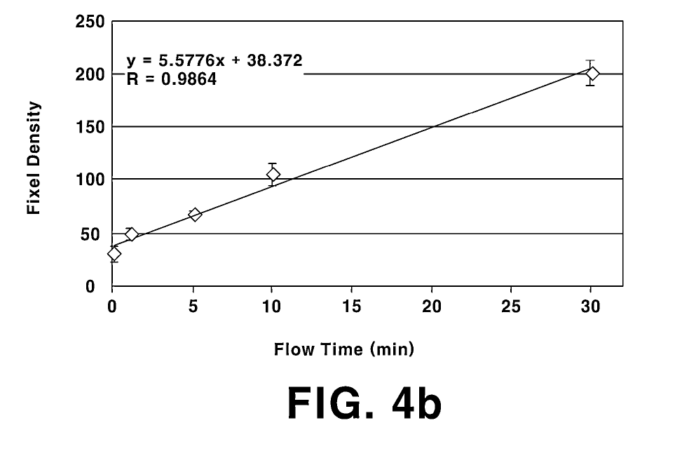

As a result, it was verified that the fluorescence intensity linearly increased in proportion to the amount (or time) of mercury ion solution passing through the membrane as shown in FIGS. 4A and 4B. When the flow rate of the pump is equal, it can be estimated that there is a proportional correlation between the gradient of the fluorescence signal increase according to the time and the mercury ion concentration of the sample.

In order to verify the continuous monitoring possibility for mercury ion using a signal accumulation-based porous membrane unit based on the results acquired above, when the receptor complex was fixed on the silica membrane and then distilled water and mercury ion solution with different concentrations were passed through the membrane, the change of the fluorescence signal intensity on the surface of the membrane was analyzed.

The flow rate was fixed at about 5 ml/min, and each sample was passed through the membrane in the order of distilled water, 0.5 ppm mercury ion solution, distilled water, 1 ppm mercury ion solution, distilled water, 0.5 ppm mercury ion solution, and distilled water.

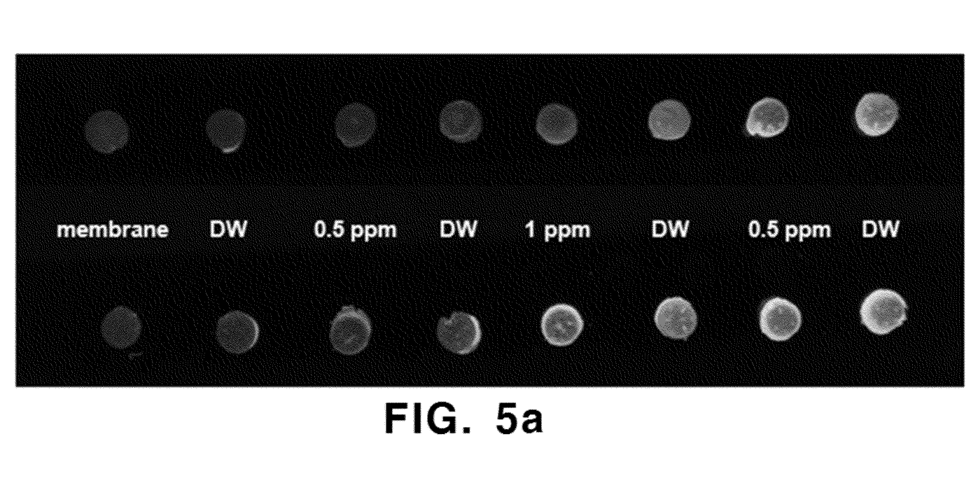

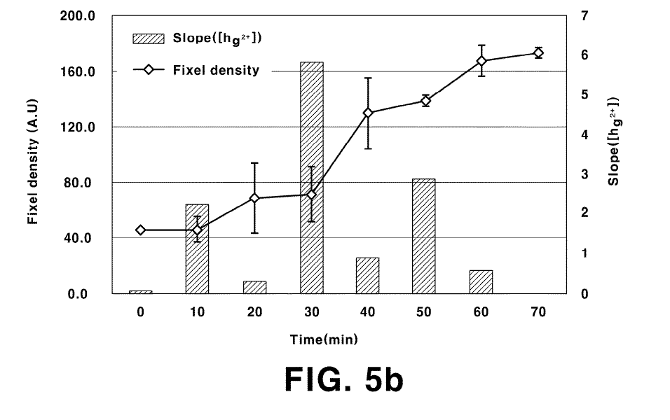

As shown in FIGS. 5A and 5B, when distilled water was passed, the change of the fluorescence signal was very insignificant, and when mercury ion solution was passed, the intensity of the fluorescence signal increased. Also, the increase speed of the fluorescence intensity was proportional to the concentration of the mercury ion in the sample.

Thus, when passing through the membrane, the concentration of the mercury ion of the aqueous sample at the moment of passing through the membrane can be expressed as a function of the increase speed of the fluorescence signal that is accumulated. Accordingly, it could be verified that the mercury ion concentration of the sample at each time can be traced and monitored through the continuous measurement of the fluorescence intensity accumulation value and the analysis of the change speed of the fluorescence intensity.

According to an embodiment of the present invention, an apparatus and method for continuously monitoring subaqueous target harmful substances can continuously measure the concentration of subaqueous target harmful substances using a receptor that can selectively recognize the target harmful substances, a porous membrane fixed with the receptor, and a sensing unit that continuously measures the intensity of fluorescent signals of the target harmful substance reacting with the receptor.

Also, the apparatus and method for continuously monitoring the subaqueous target harmful substances can be utilized as various apparatuses and methods for continuously sensing various harmful substances necessary to continuously monitor for the management of the water quality.

Furthermore, since the environmental issue is globally emerging as an increasingly important issue and the domestic water management standards are being continuously tightened, the apparatus and method according to an embodiment of the present invention can complement measurement apparatuses based on electrochemical analysis and existing expensive instrumental analysis apparatuses.

The invention has been described in detail with reference to exemplary embodiments thereof. However, it will be appreciated by those skilled in the art that changes may be made in these embodiments without departing from the principles and spirit of the invention, the scope of which is defined in the appended claims and their equivalents.

What is claimed is:

1. An apparatus for continuously monitoring subaqueous target harmful substances, comprising:
a porous membrane mounted in a hollow column tube;
a receptor fixed on the porous membrane and reacting with the subaqueous target harmful substances; and
a sensing unit continuously measuring a signal generated and accumulated by the reaction between the receptor and the target harmful substances.

2. The apparatus of claim 1, wherein the hollow column tube comprises an inlet connected to a container storing an aqueous sample via a tube.

3. The apparatus of claim 1, further comprising a flow rate control pump that supplies a sample solution to the membrane inside the hollow column tube at a certain flow rate.

4. The apparatus of claim 1, wherein the sensing unit is configured to sense one selected from a fluorescence signal, an electrochemical signal, an electric signal, and an optical signal such as a color change, which are generated and accumulated by the reaction between the receptor and the target harmful substances.

5. The apparatus of claim 1, wherein the porous membrane is manufactured by cutting a material selected from silica, cellulose, polymer, and metal foam.

6. The apparatus of claim 1, wherein the receptor is capable of selectively recognize a biomaterial comprising a functional DNA, an aptamer, an antibody and an enzyme, and a specific subaqueous target substance of a functional polymer, an inorganic material and organic/inorganic materials

7. The apparatus of claim 1, wherein when a mercury ion that is one of main indicators for water quality management is selected as the target substance, a functional single-stranded DNA capable of selectively combining with the mercury ion is selected as the receptor.

8. The apparatus of claim 1, wherein the receptor is a receptor complex in which a single-stranded DNA receptor and a complementary DNA having a complementary nucleotide sequence with respect to the single-stranded DNA receptor are combined in a double helix structure.

9. The apparatus of claim 8, wherein the complementary DNA has a fluorescent material and an amine group functionalized at both ends thereof, respectively, the fluorescent material emitting a fluorescence and the amine group being for a fixation on the membrane.

10. The apparatus of claim 8, wherein the single-stranded DNA receptor has one end marked with an inhibiting substance capable of a fluorescence signal.

11. A method for continuously monitoring subaqueous target harmful substances, comprising:
manufacturing a receptor complex in which a target substance selective DNA receptor and a complementary DNA complementarily combining with the target substance selective DNA receptor are combined in a double helix structure;
fixing the receptor complex on a porous membrane;
continuously passing a solution containing a target substance through the porous membrane on which the receptor complex is fixed and then continuously measuring a fluorescence signal generated by a reaction between the receptor and the target substance on a surface of the membrane; and
detecting an instantaneous concentration of the target substance in the sample solution that is introduced, by analyzing a variation of the fluorescence signal that is continuously measured.

12. The method of claim 11, wherein the surface of the membrane is chemically functionalized before the receptor complex is fixed on the porous membrane.

13. The method of claim 11, wherein the surface of the membrane on which the receptor complex is not fixed is treated with a blocking solution.

14. The method of claim 11, wherein before a reaction between the receptor complex and the target substance, a fluorescence emitted from a fluorescent material marked on an end of the complementary DNA of the receptor complex is inhibited from emitting to the outside by an inhibiting material marked on an end of the receptor DNA.

15. The method of claim 11, wherein when the solution containing the target substance is continuously passed through the porous membrane, the target substance combines with the receptor DNA, and simultaneously, the receptor DNA is separated from the complementary DNA due to a flow force of the target substance, enabling a fluorescence expression of a fluorescent material on an end of the complementary DNA.

16. The method of claim 11, wherein in the continuous measuring of the fluorescence signal, the fluorescence signal according to a fluorescence expression of a fluorescent material of the complementary DNA is accumulated and increased in proportion to an amount of the target substance passing through the membrane, and an increase speed of the fluorescence signal increases in proportion to a concentration of the target substance.

17. The method of claim 16, wherein when an intensity of the fluorescence signal is saturated, the membrane and the receptor are replaced.


SEQUENCE LISTING (2 sequences)

SEQ ID NO: 1 - DNA, 21 bp, Artificial Sequence
  R-DNA
ttctttcttcccttgtttgtt

SEQ ID NO: 2 - DNA, 21 bp, Artificial Sequence
  C-DNA
aagaaagaagggaacaaacaa